(19) United States
(12) Patent Application Publication
(10) Pub. No.: US 2022/0250074 A1
(43) Pub. Date: Aug. 11, 2022

(54) MESH FOR CELL LAYER PREPARATION

(71) Applicant: Orca Biosystems, Inc., Menlo Park, CA (US)

(72) Inventors: Qiong PAN, Hayward, CA (US); Syed HUSAIN, Fremont, CA (US); Lagnajeet PRADHAN, Union City, CA (US); Efrat HAREL, Menlo Park, CA (US); Ivan K. DIMOV, Los Altos Hills, CA (US); Colm HUNT, Redwood City, CA (US); Wesley DONG, Belmont, CA (US)

(21) Appl. No.: 17/689,334

(22) Filed: Mar. 8, 2022

Related U.S. Application Data
(63) Continuation of application No. PCT/US2020/050004, filed on Sep. 9, 2020.
(60) Provisional application No. 62/898,357, filed on Sep. 10, 2019.

(51) Int. Cl. B01L 3/00

(52) U.S. Cl. CPC B01L 3/502761; B01L 2200/0652; B01L 2400/0487; B01L 2300/123; B01L 2400/0406; B01L 2300/0681

(57) ABSTRACT

Methods and devices for creating monolayer arrays of cells or particles are described which may be used for high-throughput cell sorting and analysis, or other particle sorting applications. A cell loading system is described, the system comprising a porous mesh having a plurality of openings arranged in a random or repeating pattern across a surface. The porous mesh is used for preparing a layer of target particles, e.g., cells, distributed and spaced apart in a two-dimensional configuration on or within the mesh. Each of the plurality of openings in the mesh is configured to receive and permit a target particle to pass through when a fluid containing the target particles is dispensed on the surface of the mesh.

### CROSS-REFERENCE

This application is a continuation of International Application No. PCT/US2020/050004, filed Sep. 9, 2020, which claims the benefit of U.S. Provisional Application Ser. No. 62/898,357, filed on Sep. 10, 2019, each of which are incorporated by reference herein for all purposes.

### BACKGROUND

A cell sorting device, for example as described in U.S. Patent Application Publication No. 2018/0353960 A1, may comprise a plurality of microchannels for receiving a plurality of cells or particles therein as a fluid comprising a cell or particle suspension is dispensed onto the device. The fluid may be flown over the device in a planar configuration. During transfer of the cells into the microchannels, some of the cells may not locate to a desired position within the microchannels. For example, a cell may be located and affixed at a proximal end instead of a distal end of the microchannel. In some instances, a microchannel may be cluttered with two or more cells, which may affect subsequent release of cells from the microchannels. In other instances, a microchannel may not contain any cell due to clustering of cells at an opening of the microchannel. Accordingly, there is a need for methods and devices that can generate a monolayer array of distributed cells or particles for use with a cell sorting device that can improve yield and reliability in high-throughput cell sorting and analysis, or other particle sorting applications.

### SUMMARY

The devices and method described herein can address at least the above stated need.

Disclosed herein are particle loading system comprising: a porous mesh having a plurality of openings arranged in a repeating pattern across a surface of said mesh, wherein said surface comprises a first surface and a second surface opposite to the first surface, wherein the mesh is used for preparing a layer of target particles distributed and spaced apart in a two-dimensional configuration on said mesh, and wherein each of the plurality of openings of the mesh is configured to receive and permit a target particle to pass through when a fluid containing the target particles is dispensed on the first surface of the mesh.

In some embodiments, the repeating pattern comprises a cross-hatch pattern. In some embodiments, each of the plurality of openings is configured to receive and permit no more than one target particle to pass through from the first surface to the second surface of the mesh in a given instance. In some embodiments, the layer of target particles is held together by surface tension between the fluid and the plurality of openings. In some embodiments, the layer of target particles comprises one or more monolayers of cells distributed and spaced apart in the two-dimensional configuration on the mesh. In some embodiments, the system further comprises: a fluid distribution apparatus configured to come in contact with the second surface of the mesh, wherein said contact distributes the fluid over distal ends of the plurality of openings to aid the preparation of the layer of target particles. In some embodiments, the fluid distribution apparatus is configured to cause the layer of target particles to be held together in a layer of the fluid. In some embodiments, the fluid distribution apparatus is configured to translate across the second surface of the mesh from one end of the mesh to an opposite end of the mesh. In some embodiments, the second surface of the mesh is configured to translate along the fluid distribution apparatus from one end of the mesh to an opposite end of the mesh. In some embodiments, a contact force between the fluid distribution apparatus and the second surface of the mesh ranges from about 0.01 N to about 0.03 N. In some embodiments, the fluid distribution apparatus comprises a spreading element selected from the group consisting of a semi-cylindrical roller, a cylindrical roller, and a squeegee blade. In some embodiments, the mesh is made of a flexible material. In some embodiments, the mesh is held in tension by a support frame when the fluid containing the target particles is dispensed on the first surface of the mesh. In some embodiments, the mesh is capable of flexing by different amounts, depending on (1) a volume of the fluid dispensed on the first surface of the mesh and (2) the tension within the mesh as provided by the support frame. In some embodiments, the system further comprises: one or more dispense ports coupled to the support frame and configured to dispense the fluid containing the target particles at one or more edges of the mesh. In some embodiments, the one or more dispense ports are configured to dispense the fluid containing the target particles at a selected edge of the mesh. In some embodiments, the mesh is configured to be tilted at an inclination angle when the fluid containing the target particles is dispensed on the mesh. In some embodiments, the mesh is tilted at the inclination angle to cause the fluid containing the target particles to move across the mesh via capillary action against gravity. In some embodiments, the movement of the fluid across the mesh via the capillary action aids in reducing air bubbles within the layer of the target particles. In some embodiments, the inclination angle is defined between the selected edge of the mesh and a horizontal plane, and ranges from about 2 degrees to about 10 degrees. In some embodiments, each of the plurality of openings has a diameter of about 30 μm. In some embodiments, the openings within the plurality of openings are spaced apart by an edge-to-edge distance of about 0.01 mm to 1 mm. In some embodiments, the mesh is configured to be placed in proximity to a cassette used for detecting and sorting the target particles. In some embodiments, the second surface of the mesh is configured to align with the cassette. In some embodiments, the system further comprises: a transfer apparatus configured to transfer the layer of target particles from the mesh to a plurality of microchannels within the cassette. In some embodiments, the transfer apparatus comprises a vacuum suction element.

Also disclosed herein are particle loading methods comprising: providing a porous mesh having a plurality of openings arranged in a repeating pattern across a surface of said mesh, wherein said surface comprises a first surface and a second surface opposite to the first surface; and dispensing a fluid containing a plurality of target particles on the first surface of the mesh, thereby causing said fluid containing said target particles to flow across said mesh to form a layer of target particles distributed and spaced apart in a two-dimensional configuration on said mesh, wherein each of the plurality of openings of the mesh is configured to receive and permit a target particle to pass through. In some embodiments, the target particles are cells.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic cross-sectional illustration of a porous mesh device of the present disclosure.

FIG. 2A shows a photograph of a prototype mesh device comprising a support frame.

FIG. 2B shows different exemplary patterns of the pores in the porous mesh device.

FIG. 3 illustrates components of a microchannel plate device for sorting cells or particles, as described in U.S. Patent Application Publication No. 2018/0353960 A1.

FIG. 4 illustrates the use of a porous mesh device of the present disclosure to separate cells into a monolayer which may then be transferred to a microchannel plate device.

FIG. 5 illustrates a single cell suspended in the fluid within a microchannel.

FIG. 6 illustrates the use of a porous mesh device of the present disclosure to separate cells.

FIG. 7 represents the performance of the porous mesh devices of the present disclosure.

### DETAILED DESCRIPTION

Mesh-based devices for preparing layers of individual cells or other particles, and methods and systems for their use, are described herein. In some instances, the device may comprise a porous, substantially planar mesh (or membrane) having a plurality of pores or openings arranged in a random or repeating pattern across its surface, where the pores span the thickness of the mesh and are in fluid communication with a top (or first) surface and a bottom (or second) surface of the mesh. The mesh may be loaded with a fluid comprising a cell suspension or other particle suspension by dispensing and/or spreading the fluid across the top surface of the mesh and allowing capillary action to fill the pores. In some instances, the dimension of the pores may be sized such that only a single cell or particle may pass through the pore. As a result of the wicking of the cell or particle suspension into the plurality of pores of the mesh device, a two-dimensional layer of separated, spaced-apart, individual cells or particles is formed on or within the bottom surface of the mesh in a distributed manner. In some instances, the layer of separated, spaced-apart, individual cells or particles formed may comprise a two-dimensional monolayer of separated, spaced-apart single cells or particles. In some instances, the layer of separated, spaced-apart individual cells or particles may be subsequently deposited on or transferred to a substrate, e.g., a planar glass or polymer substrate, for use in cell or particle imaging and analysis applications, e.g., cell morphology studies, or cell surface biomarker studies. In some instances, the mesh device may be used as a pre-screening device for any cells or particles, and the layer of separated, spaced-apart cells or particles may subsequently be transferred from the mesh into a cell or particle sorting apparatus, e.g., as described in U.S. Patent Application Publication No. 2018/0353960 A1.

As noted, this disclosure provides methods, devices, methods, and systems for separating cells or other particles into two-dimensional arrays for use in sorting and analysis applications. In some instances, the disclosed methods, devices, methods, and systems may be used for separating cells or other particles into “2.5-dimensional” arrays, e.g., where the mesh has a “2.5D” surface topology such that, e.g., alternating rows or columns of cells or particles are slightly displaced in a vertical direction relative to the plane of the substantially two-dimensional array. Various aspects of the methods, devices, and systems described herein may be applied to any of the particular applications set forth below, or to any other types of related applications known to those of skill in the art. It shall be understood that different aspects of the disclosed methods, devices, and systems can be appreciated individually, collectively, or in combination with each other.

Definitions: Unless otherwise defined, all of the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the term ‘about’ a number refers to that number plus or minus 10% of that number. The term ‘about’ when used in the context of a range refers to that range minus 10% of its lowest value and plus 10% of its greatest value.

Cells & other particles: As used herein, the term “cell” may refer to any of a variety of cells known to those of skill in the art. In some aspects, the term “cell” may refer to any adherent or non-adherent eukaryotic cell, mammalian cell, a primary or immortalized human cell or cell line, a primary or immortalized rodent cell or cell line, a cancer cell, a normal or diseased human cell derived from any of a variety of different organs or tissue types (e.g., a white blood cell, red blood cell, platelet, epithelial cell, endothelial cell, neuron, glial cell, astrocyte, fibroblast, skeletal muscle cell, smooth muscle cell, gamete, or cell from the heart, lungs, brain, liver, kidney, spleen, pancreas, thymus, bladder, stomach, colon, small intestine), a distinct cell subset such as an immune cell, a CD8⁺ T cell, CD4⁺ T cell, CD44^{high}/CD24^{low} cancer stem cell, Lgr5/6⁺ stem cell, undifferentiated human stem cell, a human stem cell that has been induced to differentiate, a rare cell (e.g., a circulating tumor cell (CTC), a circulating epithelial cell, a circulating endothelial cell, a circulating endometrial cell, a bone marrow cell, a progenitor cell, a foam cell, a mesenchymal cell, or a trophoblast), an animal cell (e.g., mouse, rat, pig, dog, cow, or horse), a plant cell, a yeast cell, a fungal cell, a bacterial cell, an algae cell, an adherent or non-adherent prokaryotic cell, or in plural form, any combination thereof. In some aspects, the term “cell” may refer to an immune cell, e.g., a T cell, a cytotoxic (killer) T cell, a helper T cell, an alpha beta T cell, a gamma delta T cell, a T cell progenitor, a B cell, a B-cell progenitor, a lymphoid stem cell, a myeloid progenitor cell, a lymphocyte, a granulocyte, a Natural Killer cell, a plasma cell, a memory cell, a neutrophil, an eosinophil, a basophil, a mast cell, a monocyte, a dendritic cell, and/or a macrophage, or in plural form, to any combination thereof.

In some instances, the disclosed methods, devices, and systems may be used for separating and creating two-dimensional layers of particles other than cells. Examples include, but are not limited to, lipid vesicles, extracellular vesicles, microparticles, microbeads, chemical synthesis resin particles, glass microspheres, polymer microspheres, metal microspheres, ceramic microspheres, and the like, or any combination thereof.

In some instances, the average diameter or dimension of the cells or particles for which the disclosed methods and devices may be used may range from about 0.5 μm to about 0.5 mm. In some instances, the average diameter or dimension of the cells or particles may be at least 0.5 μm, at least 1 μm, at least 2 μm, at least 3 μm, at least 4 μm, at least 5 μm, at least 6 μm, at least 7 μm, at least 8 μm, at least 9 μm, at least 10 μm, at least 20 μm, at least 30 μm, at least 40 μm, at least 50 μm, at least 60 μm, at least 70 μm, at least 80 μm, at least 90 μm, at least 0.1 mm, at least 0.2 mm, at least 0.3 mm, at least 0.4 mm, or at least 0.5 mm. In some instances, the average diameter or dimension of the cells or particles may be at most 0.5 mm, at most 0.4 mm, at most 0.3 mm, at most 0.2 mm, at most 0.1 mm, at most 90 μm, at most 80 μm, at most 70 μm, at most 60 μm, at most 50 μm, at most 40 μm, at most 30 μm, at most 20 μm, at most 10 μm, at most 9 μm, at most 8 μm, at most 7 μm, at most 6 μm, at most 5 μm, at most 4 μm, at most 3 μm, at most 2 μm, at most 1 μm, or at most 0.5 μm. Any of the lower and upper values described in this paragraph may be combined to form a range included within the present disclosure, for example, in some instances the average diameter or dimension of the cells or particles may range from about 5 μm to about 40 μm. In some instances, the average diameter or dimension of the cells or particles may have any value within this range, e.g., about 12.4 μm.

Mesh Material and Porosity:

The disclosed devices comprise a porous mesh or membrane that may be fabricated from any of a variety of flexible or rigid materials known to those of skill in the art (e.g., comprising a Young's modulus ranging from about 0.1×10⁶ N/cm² to about 200×10⁶ N/cm², or higher). In some instances, the mesh may be fabricated from a material comprising a Young's modulus of at least 0.1×10⁶ N/cm², at least 0.5×10⁶ N/cm², at least 1×10⁶ N/cm², at least 5×10⁶ N/cm², at least 10×10⁶ N/cm², at least 50×10⁶ N/cm², at least 100×10⁶ N/cm², at least 150×10⁶ N/cm², or at least 200×10⁶ N/cm². In some instances, the choice of material may depend on a property of the material, e.g., hydrophilicity, hydrophobicity, or chemical resistance. In some instances, the choice of material used may depend on the choice of fabrication technique, and vice versa. In some instances, the mesh may be fabricated from, e.g., glass, silicon, ceramic, metal (e.g., a stainless-steel mesh), carbon fibers, or a polymer material. Examples of suitable polymer materials include, but are not limited to, polydimethylsiloxane (PDMS; elastomer), polymethylmethacrylate (PMMA), polycarbonate (PC), polystyrene (PS), polypropylene (PP), polyethylene (PE), polyfluorinated polyethylene, high density polyethylene (HDPE), polyether ether ketone, polyimide, cyclic olefin polymers (COP), cyclic olefin copolymers (COC), polyethylene terephthalate (PET), epoxy resins, a non-stick material such as teflon (polytetrafluoroethylene (PTFE)), a photoresist such as SU8 or any other thick film photoresist, or any combination of these materials. Examples of suitable metal mesh materials include, but are not limited to, stainless steel, aluminum, or copper.

The porous mesh or membrane may be fabricated using any of a variety of techniques known to those of skill in the art provided that they are compatible with the feature dimensions of the porous mesh design and the material from which the porous mesh is fabricated. Example of suitable fabrication techniques include, but are not limited to, weaving, polymer micro-molding using a silicon or metal master that has been photolithographically-patterned and etched; three-dimensional (3D) printing; photolithograpic patterning and wet chemical etching, dry etching, deep reactive ion etching, laser micromachining, and the like.

In some instances, the porous mesh may comprise any of a variety of regular or irregular (amorphous) shapes in two dimensions, e.g., circular, square, rectangular, triangular, pentagonal, hexagonal, and so forth. In some instances, the diameter or longest dimension, such as length or width, of the porous mesh may range from about 1 cm to about 40 cm. In some instances, the diameter or longest dimension of the porous mesh may be at least 1 cm, at least 2 cm, at least 3 cm, at least 4 cm, at least 5 cm, at least 6 cm, at least 7 cm, at least 8 cm, at least 9 cm, at least 10 cm, at least 15 cm, at least 20 cm, at least 25 cm, at least 30 cm, at least 35 cm, or at least 40 cm. In some instances, the diameter or longest dimension of the porous mesh may be at most 40 cm, at most 35 cm, at most 30 cm, at most 25 cm, at most 20 cm, at most 15 cm, at most 10 cm, at most 9 cm, at most 8 cm, at most 7 cm, at most 6 cm, at most 5 cm, at most 4 cm, at most 3 cm, at most 2 cm, or at most 1 cm. Any of the lower and upper values described in this paragraph may be combined to form a range included within the present disclosure, for example, in some instances the diameter or longest dimension of the porous mesh may range from about 3 cm to about 15 cm. In some instances, the diameter or longest dimension of the porous mesh may have any value within this range, e.g., about 18.5 cm.

In some instances, the porous mesh may have a thickness ranging from about 0.01 mm to about 10 mm. In some instances, the porous mesh may have a thickness ranging from about 0.01 mm to about 1 mm. In some instances, the thickness of the porous mesh may be at least 0.01 mm, at least 0.05 mm, at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1.0 mm, at least 2.0 mm, at least 3.0 mm, at least 4.0 mm, at least 5.0 mm, at least 6.0 mm, at least 7.0 mm, at least 8.0 mm, at least 9.0 mm, or at least 10.0 mm. In some instances, the thickness of the porous mesh may be at most 10.0 mm, at most 9.0 mm, at most 8.0 mm, at most 7.0 mm, at most 6.0 mm, at most 5.0 mm, at most 4.0 mm, at most 3.0 mm, at most 2.0 mm, at most 1.0 mm, at most 0.9 mm, at most 0.8 mm, at most 0.7 mm, at most 0.6 mm, at most 0.5 mm, at most 0.4 mm, at most 0.3 mm, at most 0.2 mm, at most 0.1 mm, at most 0.05 mm, or at most 0.01 mm. Any of the lower and upper values described in this paragraph may be combined to form a range included within the present disclosure, for example, in some instances the thickness of the porous mesh may range from about 0.2 mm to about 0.8 mm. In some instances, the thickness of the porous mesh may range from about 0.5 mm to about 5.0 mm. In some instances, the thickness of the porous mesh may have any value within this range, e.g., about 0.15 mm.

In some instances, the porous mesh of the disclosed devices may comprise any random or non-random pattern of pores or openings across the surface of the mesh. Examples of non-random patterns of pores or openings include, but are not limited to, a square grid, a rectangular grid, a triangular grid, a hexagonal grid, a cross-hatch, or any other pattern or distribution of pores or opening. In some instances, the number of pores or openings per unit area of mesh may range from about 1 per mm² to about 200 per mm². In some instances, the number of pores or openings per unit area of mesh may be at least 1 per mm², at least 10 per mm², at least 20 per mm², at least 30 per mm², at least 40 per mm², at least 50 per mm², at least 60 per mm², at least 70 per mm², at least 80 per mm², at least 90 per mm², at least 100 per mm², at least 120 per mm², at least 140 per mm², at least 160 per mm², at least 180 per mm², or at least 200 per mm². In some instances, the number of pores or openings per unit area of mesh may be at most 200 per mm², at most 180 per mm², at most 160 per mm², at most 140 per mm², at most 120 per mm², at most 100 per mm², at most 90 per mm², at most 80 per mm², at most 70 per mm², at most 60 per mm², at most 50 per mm², at most 40 per mm², at most 30 per mm², at most 20 per mm², at most 10 per mm², or at most 1 per mm². Any of the lower and upper values described in this paragraph may be combined to form a range included within the present disclosure, for example, in some instances the number of pores or opening per unit area of mesh may range from about 10 per mm² to about 160 per mm². In some instances, the number of pores or opening per unit area of mesh may have any value within this range, e.g., about 104 per mm². In some instances, the number of pores or opening per unit area of mesh may vary across the surface of the mesh or membrane.

In some instances, the pores or opening in the mesh may comprise any of a variety of cross-sectional shapes in two dimensions, e.g., circular, square, rectangular, triangular, pentagonal, hexagonal, and so forth. In some instances, the diameter or longest cross-sectional dimension of the pores or openings may range from about 5 μm to about 500 μm. In some instances, the diameter or longest cross-sectional dimension of the pores or openings may be at least 5 μm, at least 10 μm, at least 15 μm, at least 20 μm, at least 25 μm, at least 30 μm, at least 35 μm, at least 40 μm, at least 45 μm, at least 50 μm, at least 75 μm, at least 100 μm, at least 200 μm, at least 300 μm, at least 400 μm, or at least 500 μm. In some instances, the diameter or longest cross-sectional dimension of the pores or opening may be at most 500 μm, at most 400 μm, at most 300 μm, at most 200 μm, at most 100 μm, at most 75 μm, at most 50 μm, at most 45 μm, at most 40 μm, at most 35 μm, at most 30 μm, at most 25 μm, at most 20 μm, at most 15 μm, at most 10 μm, or at most 5 μm. Any of the lower and upper values described in this paragraph may be combined to form a range included within the present disclosure, for example, in some instances the diameter or longest cross-sectional dimension of the pores or opening may range from about 10 μm to about 200 μm. In some instances, the diameter or longest cross-sectional dimension of the pores or openings may have any value within this range, e.g., about 28.5 μm. In some instances, the diameter or longest cross-sectional dimension of the pores or openings may vary across the surface of the mesh or membrane. In some instances, the mesh or membrane may comprise 1, 2, 3, 4, or 5, or more subsets of pores or openings having different diameters or longest cross-sectional dimensions. In some instance, the surface tension of the fluid in which particles, e.g., beads, are suspended may not initially be strong enough to form a meniscus at the bottom of a pore if the pore diameter is too large. In these instances, further optimization of the fluid composition to modify its surface tension and/or other fluid properties may allow the use of larger pore diameters. In some instances, the mesh 110 may be partially sealed or coated. For example, at least one edge of the mesh 110 may be sealed. In some instances, the parameter of the mesh is partially sealed. In some instances, the mesh 110 is not partially sealed. In some instances, the center portion of the mesh 110 is not partially sealed.

In some instances, the center-to-center spacing (or “pitch”) or edge-to-edge separation distance between adjacent pores or openings may range from about 5 μm to about 500 μm. In some instances, the center-to-center spacing (or “pitch”) or edge-to-edge separation distance may be at least 5 μm, at least 10 μm, at least 15 μm, at least 20 μm, at least 25 μm, at least 30 μm, at least 35 μm, at least 40 μm, at least 45 μm, at least 50 μm, at least 75 μm, at least 100 μm, at least 200 μm, at least 300 μm, at least 400 μm, or at least 500 μm. In some instances, the center-to-center spacing (or “pitch”) or edge-to-edge separation distance may be at most 500 μm, at most 400 μm, at most 300 μm, at most 200 μm, at most 100 μm, at most 75 μm, at most 50 μm, at most 45 μm, at most 40 μm, at most 35 μm, at most 30 μm, at most 25 μm, at most 20 μm, at most 15 μm, at most 10 μm, or at most 5 μm. Any of the lower and upper values described in this paragraph may be combined to form a range included within the present disclosure, for example, in some instances the center-to-center spacing (or “pitch”) or edge-to-edge separation distance may range from about 20 μm to about 75 μm. In some instances, the center-to-center spacing (or “pitch”) or edge-to-edge separation distance may have any value within this range, e.g., about 12.5 μm. In some instances, the center-to-center spacing (or “pitch”) or edge-to-edge separation distance between adjacent pores or openings may vary across the surface of the mesh or membrane.

In general, the length (or height) of the pores or openings may be equal to the thickness of the mesh or membrane. In some instances, the cross-sectional area of the pores or openings may be constant over their entire length. In some instances, the cross-sectional area of the pores or openings may vary their length. For example, in some instances, the entrance and/or exit of the pore or opening may be tapered such that the cross-sectional area of the pore at the top surface and/or bottom surface of the mesh is larger than that at the mid-point of the mesh.

Support Frame Design and Fabrication:

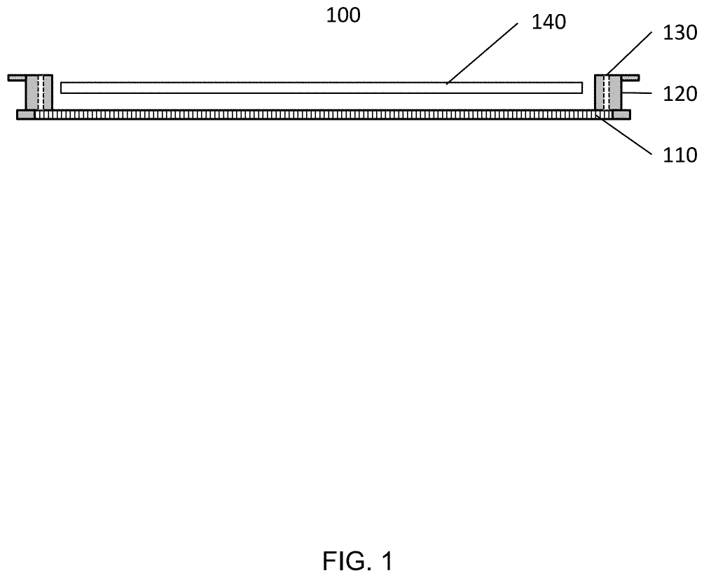

FIG. 1 provides a cross-sectional illustration of a porous mesh device 100 of the present disclosure. In some instances, the porous mesh 110 may be mounted in a rigid or semi-rigid support frame 120 such as that illustrated in FIG. 1. The frame provides for convenient handling of the porous mesh and facilitates the alignment and interfacing of the device with other cell or particle sorting and analysis apparatus. In some instances, the support frame may be designed to maintain a specified level of tension in, e.g., a flexible polymer mesh mounted in the rigid frame. In some instances, the tension applied to the mesh may range from about 10 Newtons to about 300 Newtons. In some instances, the applied tension may be at least 10, at least 20, at least 30, at least 40, at least 50, at least 60, at least 70, at least 80, at least 90, at least 100, at least 120, at least 140, at least 160, at least 180, at least 200, at least 220, at least 240, at least 260, at least 280, or at least 300 Newtons. In some instances, the applied tension may be at most 300, at most 280, at most 260, at most 240, at most 220, at most 200, at most 180, at most 160, at most 140, at most 120, at most 100, at most 90, at most 80, at most 70, at most 60, at most 50, at most 40, at most 30, at most 20, or at most 10 Newtons. Any of the lower and upper values described in this paragraph may be combined to form a range included within the present disclosure, for example, in some instances the applied tension may range from about 30 Newtons to about 180 Newtons. Those of skill in the art will recognize that in some instances, the applied tension may have any value within this range, e.g., about 126 Newtons.

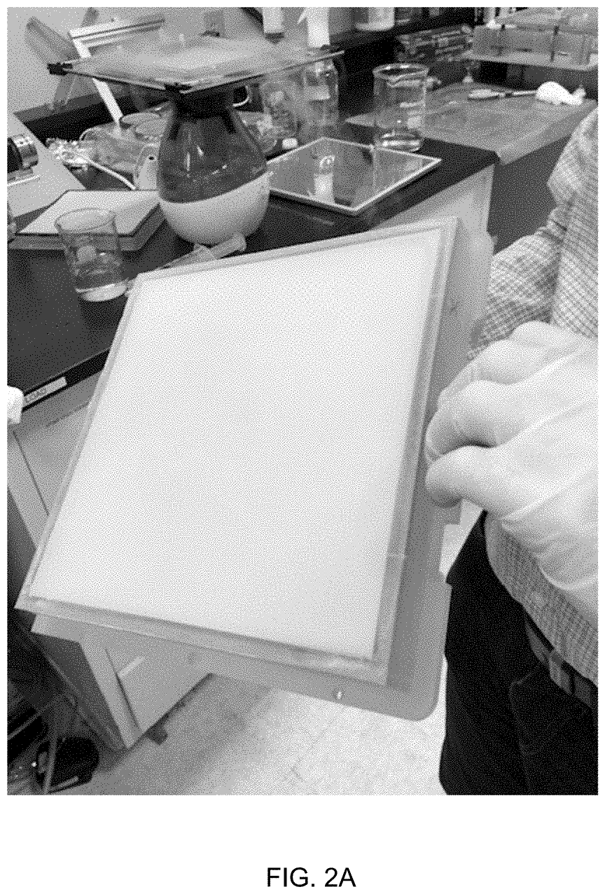

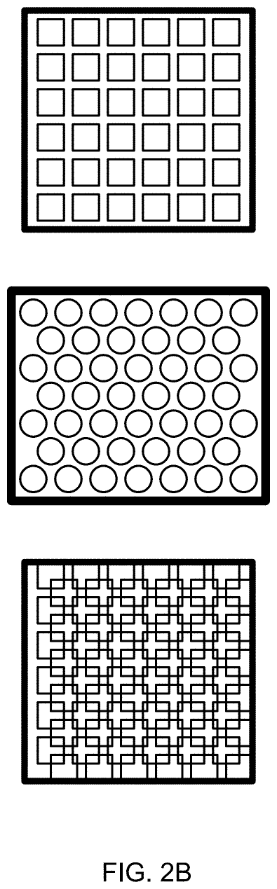

In some instances, the support frame may comprise a tensioning mechanism for adjusting or controlling the tension of the mesh. Examples of tensioning mechanisms include, but are not limited to, screws, springs, etc. In some instances, the support frame may be configured to apply uniform tension across mesh. In some instances, the tension applied by the support frame may slightly affect the pore size (e.g., through stretching of the pores) and/or the planarity of the mesh. In some instances, the support frame may comprise two or more dispensing ports 130 into which a cell suspension or particle suspension may be deposited such that the suspension fluid flows across the top surface of mesh, thereby filling the pores in the mesh with single cells or single particles. In some instances, the dispensing of a cell suspension or particle suspension may be performed at two or more dispensing ports simultaneously or sequentially to form a flow front traversing across the surface of the mesh. In some instances, the fluid dispense rate from each dispensing port may be at least 0.01 ml/min, at least 0.1 ml/min, at least 1 ml/min, at least 10 ml/min, at least 20 ml/min, at least 30 ml/min, at least 40 ml/min, at least 50 ml/min, at least 60 ml/min, at least 70 ml/min, at least 80 ml/min, at least 90 ml/min, at least 100 ml/min, at least 200 ml/min, at least 300 ml/min, at least 400 ml/min, or at least 500 ml/min. In some instances, the velocity of the fluid front that flows across the surface of the mesh may be at least 1 mm/sec, at least 5 mm/sec, at least 10 mm/sec, at least 20 mm/sec, at least 30 mm/sec, at least 40 mm/sec, at least 50 mm/sec, at least 60 mm/sec, at least 70 mm/sec, at least 80 mm/sec, at least 90 mm/sec, or at least 100 mm/sec. In some instances, the support frame may comprise 1, 2, 3, 4, 5, 6, or more dispensing ports 130. A photograph of a prototype porous mesh device comprising a support frame is shown in FIG. 2A. Schematic illustrations of exemplary patterns of the pores in the porous mesh device are shown in FIG. 2B (upper: square grid of square pores; middle: circular pores in a hexagonal close pack arrangement; lower: irregular grid comprising pores of irregular shape).

The support frame may be fabricated using any of a variety of materials known to those of skill in the art. Examples of suitable fabrication techniques include, but are not limited to, conventional machining, CNC machining, injection molding, 3D printing, and the like. The support frame may be fabricated using any of a variety of materials known to those of skill in the art. In general, the choice of material used will depend on the choice of fabrication technique, and vice versa. Examples of suitable materials include, but are not limited to, aluminum, stainless steel, glass, silicon, ceramic, and any of a variety of polymers, e.g. polydimethylsiloxane (PDMS; elastomer), polymethylmethacrylate (PMMA), polycarbonate (PC), polystyrene (PS), polypropylene (PP), polyethylene (PE), polyfluorinated polyethylene, high density polyethylene (HDPE), polyether ether ketone, polyimide, cyclic olefin polymers (COP), cyclic olefin copolymers (COC), polyethylene terephthalate (PET), epoxy resins, a non-stick material such as teflon (polytetrafluoroethylene (PTFE)), or any combination of these materials. In some instances, different components or layers of a support frame assembly may be fabricated from different materials.

Plunger plate: In some instances, the porous mesh devices of the present disclosure may further comprise a plate 140, as illustrated in FIG. 1, that is suspended above and covers substantially all of the exposed surface of mesh 110. In some instances, the surface tension of a fluid (e.g., a cell suspension fluid) that contacts and wets a surface of plate 140 prevents the fluid from leaking downward through mesh. In some instances, plate 140 may function as a “plunger” such that pressing down on the plate forces fluid through the mesh into a microchannel plate after the mesh device has been loaded onto the plate. Plate 140 may be fabricated from any of a variety of suitable materials known to those of skill in the art. Examples include, but are not limited to, glass, fused-silica, silicon, polycarbonate (PC), polymethylmethacrylate (PMMA), polypropylene (PE), cyclic olefin copolymer (COC), cyclic olefin polymer (COP), and the like. In some instances, plate 140 may be fabricated from an optically-transparent material. In some instances, a fixture or armature may be used to hold plate 140 at a fixed or adjustable distance above mesh 110. In some instances, the fixture may be a spacer. In some instances, the stand-off distance of plate 140 above mesh 110 may range from about 0.1 mm to about 2 mm. In some instances, the stand-off distance may be at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1.0 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, or at least 2.0 mm. In some instances, the stand-off distance may be at most 2.0 mm, at most 1.9 mm, at most 1.8 mm, at most 1.7 mm, at most 1.6 mm, at most 1.5 mm, at most 1.4 mm, at most 1.3 mm, at most 1.2 mm, at most 1.1 mm, at most 1.0 mm, at most 0.9 mm, at most 0.8 mm, at most 0.7 mm, at most 0.6 mm, at most 0.5 mm, at most 0.4 mm, at most 0.3 mm, at most 0.2 mm, or at most 0.1 mm. Any of the lower and upper values described in this paragraph may be combined to form a range included within the present disclosure, for example, in some instances the stand-off distance may range from about 0.3 mm to about 1.9 mm. Those of skill in the art will recognize that the stand-off distance may have any value within this range, e.g., about 1.45 mm.

Compressible Medium:

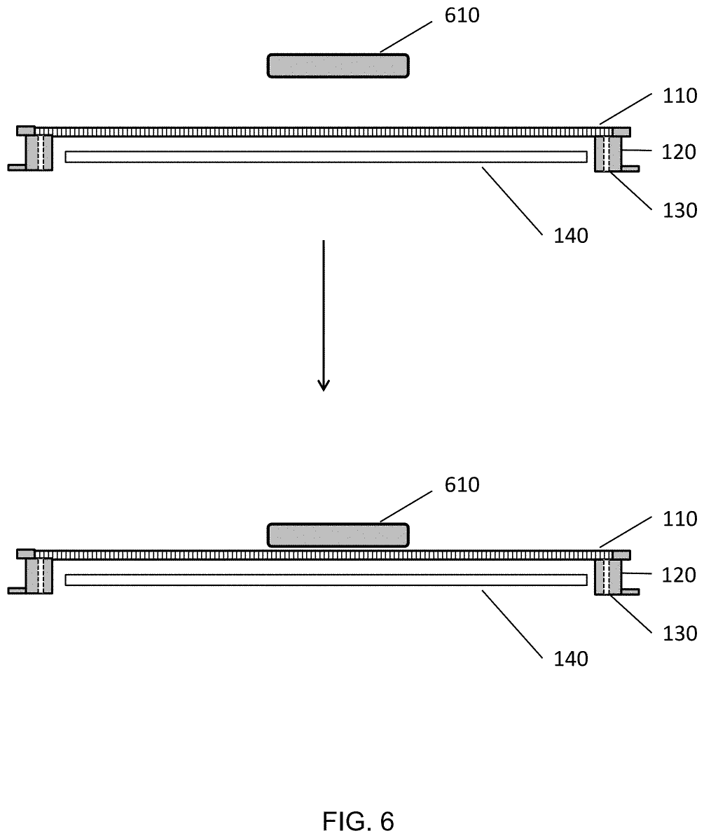

In some instances, the porous mesh devices of the present disclosure may further comprise a compressible medium 610, as illustrated in FIG. 6, that may be applied to the mesh 110. FIG. 6 illustrates the use of the compressible medium 610 on a porous mesh device of the present disclosure. In some instances, the compressible medium comprises liquid media. The compressible medium may be a soft, elastic, absorbent or porous body, such as a sponge, a cloth, and the like, that is configured to retain liquid media. In some instances, the compressible medium may transfer liquid media from the mesh onto the plate 140 with minimal or no damage to the mesh. In some instances, the transfer of liquid media from the compressible medium may flush cells onto the plate 140. In some instances, the volume of the liquid media may be changed (e.g. reduced) when pressure is applied to the compressible medium. In some instances, compression of the compressible medium may provide a fluidic flushing force that transfers the cells or particles from the mesh 110 to the plate 140. In some instances, the fluid flushing force may be about 0.02 PSI to 5 PSI. In some instances, the fluid flushing force may be at least 0.02 PSI, at least 0.04 PSI, at least 0.06 PSI, at least 0.08 PSI, at least 1.0 PSI, at least 1.2 PSI, at least 1.4 PSI, at least 1.6 PSI, at least 1.8 PSI, at least 2.0 PSI, at least 2.2 PSI, at least 2.4 PSI, at least 2.6 PSI, at least 2.8 PSI, at least 3.0 PSI, at least 3.2 PSI, at least 3.4 PSI, at least 3.6 PSI, at least 3.8 PSI, at least 4.0 PSI, at least 4.2 PSI, at least 4.4 PSI, at least 4.6 PSI, at least 4.8 PSI, or at least 5.0 PSI. In some instances, the flushing force may be at most 5.0 PSI, at most 4.8 PSI, at most 4.6 PSI, at most 4.4 PSI, at most 4.2 PSI, at most 4.0 PSI, at most 3.8 PSI, at most 3.6 PSI, at most 3.4 PSI, at most 3.2 PSI, at most 3.0 PSI, at most 2.8 PSI, at most 2.6 PSI, at most 2.4 PSI, at most 2.2 PSI, at most 2.0 PSI, at most 1.8 PSI, at most 1.6 PSI, at most 1.4 PSI, at most 1.2 PSI, at most 1.0 PSI, at most 0.8 PSI, at most 0.6 PSI, at most 0.4 PSI, or at most 0.2 PSI. Any of the lower and upper values described in this paragraph may be combined to form a range included within the present disclosure, for example, in some instances the flushing force may be about 1.0 PSI to 3.0 PSI. Those of skill in the art will recognize that in some instances, the flushing force may have any value within this range, e.g., about 2.0 PSI.

In some instances, the fluid flushing force may be uniform or variable. In some instances, the force may be controlled to transfer liquid media with minimal or no damage to the mesh 110 and plate 140. The compressible medium can be pressed against, translated and/or rolled over the mesh 110, either manually or by an automation tool (e.g. a robotic arm).

Manual and Automated Cell Loading Methods:

In some instances, the mesh device may be loaded manually, e.g., by pipetting a suspension of cells or other particles directly onto the top surface of the porous mesh, or into one or more dispensing ports that have been integrated into a support frame that holds the porous mesh. In some instances, the mesh device may be loaded automatically, e.g., using a robotic fluid dispensing system to dispense a suspension of cells or other particles directly onto the top surface of the porous mesh, or into one or more dispensing ports that have been integrated into the support frame.

In some instances, the mesh device may be loaded with a volume of about 10 to 2500 uL of a cell containing media. In some instances, the loaded volume may be at least 10 uL, at least 20 uL, at least 30 uL, at least 40 uL, at least 50 uL, at least 60 uL, at least 70 uL, at least 80 uL, at least 90 uL, at least 100 uL, at least 200 uL, at least 300 uL, at least 400 uL, at least 500 uL, at least 600 uL, at least 700 uL, at least 800 uL, at least 900 uL, at least 1000 uL, at least 1100 uL, at least 1200 uL, at least 1300 uL, at least 1400 uL, at least 1500 uL, at least 1600 uL, at least 1700 uL, at least 1800 uL, at least 1900 uL, at least 2000 uL, at least 2100 uL, at least 2200 uL, at least 2300 uL, at least 2400 uL, or at least 2500 uL. In some instances, the loaded volume may be at most 2500 uL, at most 2000 uL, at most 1900 uL, at most 1800 uL, at most 1700 uL, at most 1600 uL, at most 1500 uL, at most 1400 uL, at most 1300 uL, at most 1200 uL, at most 1100 uL, at most 1000 uL, at most 900 uL, at most 800 uL, at most 700 uL, at most 600 uL, at most 500 uL, at most 400 uL, at most 300 uL, at most 200 uL, at most 100 uL, at most 90 uL, at most 80 uL, at most 70 uL, at most 60 uL, at most 50 uL, at most 40 uL, at most 30 uL, at most 20 uL, or at most 10 uL. Any of the lower and upper values described in this paragraph may be combined to form a range included within the present disclosure, for example, in some instances the mesh device may be loaded with a volume of about 100 to 500 uL of a cell containing media. Those of skill in the art will recognize that in some instances, the loaded volume may have any value within this range, e.g., about 30 uL.

In some instances, the layer of cell containing media on the mesh has a thickness of about 1 to 100 μm. In some instances, the thickness layer may be at least 1 μm, at least 5 μm, at least 10 μm, at least 15 μm, at least 20 μm, at least 25 μm, at least 30 μm, at least 35 μm, at least 40 μm, at least 45 μm, at least 50 μm, at least 55 μm, at least 60 μm, at least 75 μm, at least 80 μm, at least 85 μm, at least 90 μm, at least 95 μm, or at least 100 μm. In some instances, the thickness layer may be at most 100 μm, at most 95 μm, at most 90 μm, at most 85 μm, at most 80 μm, at most 75 μm, at most 60 μm, at most 55 μm, at most 50 μm, at most 45 μm, at most 40 μm, at most 35 μm, at most 30 μm, at most 25 μm, at most 20 μm, at most 15 μm, at most 10 μm, at most 5 μm, or at most 1 μm. Any of the lower and upper values described in this paragraph may be combined to form a range included within the present disclosure, for example, in some instances the layer of cell containing media on the mesh may have a thickness of about 10 to 50 μm. Those of skill in the art will recognize that in some instances, the thickness layer may have any value within this range, e.g., about 20 μm.

In some instances, the filling/loading process may result in trapped air pockets or bubbles within the pores or openings in some areas of the porous mesh, for example due to non-uniform flow speeds or flow fronts. The trapped air pockets or bubbles can prevent cells or particles from entering those pores or openings of the mesh, thereby resulting in gaps within the two-dimensional monolayer of cells or particles. To improve the formation of the two-dimensional monolayer of cells or particles, the porous mesh may be tilted at a slight angle, e.g., an inclination angle, during the filling/loading process such that capillary action transports the cell or particle suspension fluid uphill and minimizes or eliminates problems with trapped air. In some instances, the inclination angle may be controlled using a tilt device that allows the mesh to be tilted at a variety of different angles. In some instances, the inclination angle may be customized based on viscosity, flow speed, etc. of the fluid. In some instances, the plane of the porous mesh (or the device comprising the porous mesh) may be tilted at an inclination angle ranging from about 0 degree to about 15 degrees relative to a level surface (or horizontal plane). In some instances, the tilt or inclination angle may be at least 0 degrees, at least 1 degree, at least 2 degrees, at least 3 degrees, at least 4 degrees, at least 5 degrees, at least 6 degrees, at least 7 degrees, at least 8 degrees, at least 9 degrees, at least 10 degrees, at least 11 degrees, at least 12 degrees, at least 13 degrees, at least 14 degrees, or at least 15 degrees. In some instances, the tilt or inclination angle may be at most 15 degrees, at most 14 degrees, at most 13 degrees, at most 12 degrees, at most 11 degrees, at most 10 degrees, at most 9 degrees, at most 8 degrees, at most 7 degrees, at most 6 degrees, at most 5 degrees, at most 4 degrees, at most 3 degrees, at most 2 degrees, at most 1 degree, or at most 0 degrees. Any of the lower and upper values described in this paragraph may be combined to form a range included within the present disclosure, for example, in some instances the tilt or inclination angle may range from about 2 degrees to about 8 degrees. In some instances, the tilt or inclination angle may have any value within this range, e.g., about 5.5 degrees. The inclination angle may be optimized to reduce or eliminate trapped air pockets or bubbles, without requiring an excessive amount of time for the fluid to travel uphill and fill the pores.

As the cell suspension or other particle suspension is dispensed onto the top surface of the porous mesh and spreads, the fluid is drawn into the pores or openings by means of capillary action. In some instances, the fluid is pushed into the pores or opening by external means, such as with a compressible medium. In some instances, one or more dimensions, e.g., the diameter, of the pores or openings, are chosen such that no more than a single cell (or single particle) may enter a given pore or opening. In some instances, one or more dimensions, e.g., the diameter, of the pores or openings, are chosen such that no more than two cells or particles, three cells or particles, or four cells or particles may enter a given pore or opening. In some instances, gravity may cause the cell(s) or particle(s) in each pore or opening to settle near the bottom surface of the porous mesh. In some instances, the cell(s) or other particle(s) may be retained within or near the pore or opening by a thin layer of fluid on the bottom surface of the mesh. In some instances, the bottom surface of the porous mesh may be gently wiped using a fluid spreading element, e.g., a semi-cylindrical roller, spherical or cylindrical roller, or a squeegee blade, to remove excess fluid from the bottom surface of the porous mesh and ensure that a monolayer of cells or particles is formed within the lower end of the pores or openings. In some instances, the cell(s) or other particle(s) may be retained within the pore or opening by the fluid meniscus at the lower entrance of the pore or opening that arises from the surface tension of the fluid filling the pore and the surface properties of the material from which the mesh is fabricated. In some instances, the surface tension of the fluid in which the cells or particles are suspended may be adjusted by adjusting its composition, e.g., through the addition of detergents or other additives. In some instances, the material used for fabrication of the porous mesh and/or the dimensions of the pores may be chosen to adjust the size and/or shape of the meniscus, e.g., to facilitate release of the entrapped cells and transfer to another substrate or device.

Systems comprising the disclosed mesh devices: Disclosed herein are systems configured to perform the disclosed methods for cell or particle separation using the disclosed porous mesh devices. In some instances, the systems may comprise: (i) one or more of the disclosed porous mesh devices, (ii) a fluid dispensing module configured to dispense a cell suspension or other particle suspension onto a surface of the porous mesh, or into one or more dispensing ports integrated with a support frame for the porous mesh device(s), (iii) a fluid distribution apparatus configured to contact the bottom surface of the porous mesh with a fluid spreading element, e.g., a semi-cylindrical (or semi-cylindrical) rod, spherical (or cylindrical) rod, or a squeegee blade, to remove excess fluid from the bottom surface of the porous mesh and/or to ensure that a monolayer of cells or particles is formed within the lower end of the pores or openings, or (iv) any combination thereof. In some instances, the disclosed systems may further comprise a transfer module or mechanism for transferring a monolayer of cells or particles formed within the pores or openings of the porous mesh device onto another substrate, or into a secondary cell sorting and imaging device such as that disclosed in U.S. Patent Application Publication No. 2018/0353960 A1. In some instances, the disclosed systems may further comprise an imaging module for imaging cells or particles either within the porous mesh itself, on a substrate to which the cells or particles have been transferred, or within a secondary cell sorting and imaging device such as that disclosed in U.S. Patent Application Publication No. 2018/0353960 A1.

Fluid Dispensing Modules:

In some instances, the disclosed systems may comprise a fluid dispensing module configured for automated dispensing of cell suspensions or other particle suspensions onto a surface of the porous mesh, or into one or more dispensing ports integrated with a support frame for the porous mesh device(s). Examples of suitable commercially-available fluid handling systems (or liquid handling systems) include, but are not limited to, the Tecan Fluent® system (Tecan Trading AG, Switzerland), the Hamilton Microlab STAR and Microlab NIMBUS systems (Hamilton, Reno, Nev.), and the Agilent Bravo Automated Liquid Handling Platform and Agilent Vertical Pipetting Station (Agilent Technologies, Santa Clara, Calif.).

Fluid Distribution Apparatus:

In some instances, the disclosed systems may comprise a fluid distribution apparatus configured to contact the bottom surface of the porous mesh with a fluid spreading element, e.g., a semi-cylindrical (or semi-cylindrical) roller, spherical (or cylindrical) roller, or a squeegee blade, to remove excess fluid from the bottom surface of the porous mesh and/or to ensure that a monolayer of cells or particles is formed within the lower end of the pores or openings. In some instances, the fluid distribution apparatus, or a fluid spreading element thereof, may be configured to translate across the bottom surface of the mesh from one end of the mesh to an opposite end of the mesh. In some instances, the bottom surface of the mesh may be configured to translate along the fluid distribution apparatus, or a fluid spreading element thereof, from one end of the mesh to an opposite end of the mesh. In some instances, the fluid distribution apparatus may be configured to perform any of several possible relative motion scenarios, e.g., (i) translation of a fluid spreading element across the mesh which is fixed in place, (ii) translation of the mesh across a fluid spreading element which is fixed in place, or (iii) translation of the both the mesh and a fluid spreading element relative to each other.

In some instances, the force applied to contact the fluid spreading element with the bottom surface of the porous mesh may range from about 0.01 N to about 0.3 N. In some instances, the contact force between the fluid spreading element and the bottom surface of the porous mesh may be at least 0.01 N, at least 0.02 N, at least 0.03 N, at least 0.04 N, at least 0.05 N, at least 0.06 N, at least 0.07 N, at least 0.08 N, at least 0.09 N, at least 0.1 N, at least 0.2 N, or at least 0.3 N. In some instances, the contact force between the fluid spreading element and the bottom surface of the porous mesh may be at most 0.3 N, at most 0.2 N, at most 0.1 N, at most 0.09 N, at most 0.08 N, at most 0.07 N, at most 0.06 N, at most 0.05 N, at most 0.04 N, at most 0.03 N, at most 0.02 N, or at most 0.01 N. Any of the lower and upper values described in this paragraph may be combined to form a range included within the present disclosure, for example, in some instances the contact force between the fluid spreading element and the bottom surface of the porous mesh may range from about 0.02 N to about 0.08 N. In some instances, the contact force between the fluid spreading element and the bottom surface of the porous mesh may have any value within this range, e.g., about 0.12 N.

Transfer Module or Mechanism:

In some instances, the disclosed systems may comprise a transfer module or mechanism for transferring a monolayer of cells or particles formed within the pores or openings of the porous mesh device onto another substrate, or into a secondary cell sorting and imaging device such as that disclosed in U.S. Patent Application Publication No. 2018/0353960 A1. In some instances, the transfer module may comprise a mechanism configured to apply pressure to a top surface of the porous mesh. In some instances, the transfer module may comprise a mechanism configured to apply a vacuum suction to the bottom surface of the porous mesh, to cause the monolayer of cells or particles on the mesh to be transferred from the mesh into a plurality of microchannels of another substrate. In some instances, the pressure applied to the top surface of the porous mesh, or the vacuum applied to the bottom surface of the porous mesh, may be at least 0.1 psi, at least 0.25 psi, at least 0.5 psi, at least 0.75 psi, at least 1 psi, at least 2 psi, at least 3 psi, at least 4 psi, at least 5 psi, at least 6 psi, at least 7 psi, at least 8 psi, at least 9 psi, or at least 10 psi.

Imaging Module:

In some instances, the disclosed systems may comprise an imaging module configured to image cells or particles either within the porous mesh itself, on a substrate to which the cells or particles have been transferred, or within a secondary cell sorting and imaging device such as that disclosed in U.S. Patent Application Publication No. 2018/0353960 A1. In some instances, the field-of-view of the images acquired using the imaging module may comprise all or a portion of the porous mesh. In some instances, the imaging may comprise intermittent or periodic imaging of all or a portion of the porous mesh before, during, or after performing a transfer of cells or particles from the porous mesh device to a substrate configured to accept the transferred cells or particles, or to a secondary cell sorting and imaging device such as that disclosed in U.S. Patent Application Publication No. 2018/0353960 A1. In some instances, the imaging may comprise acquiring UV, visible, or infrared images. In some instances, the imaging may comprise acquiring fluorescence images.

Any of a variety of imaging systems or system components may be utilized for the purpose of implementing the disclosed methods, devices, and systems. Examples include, but are not limited to, one or more light sources (e.g., light emitting diodes (LEDs), diode lasers, fiber lasers, gas lasers, halogen lamps, arc lamps, etc.), condenser lenses, objective lenses, mirrors, filters, beam splitters, prisms, image sensors (e.g., CCD image sensors or cameras, CMOS image sensors or cameras), and the like, or any combination thereof. Depending on the imaging mode utilized, the light source and image sensor may be positioned on opposite sides of the porous mesh, e.g., so that absorbance-based images may be acquired. In some instances, the light source and image sensor may be positioned on the same side of the porous mesh device, e.g., so that epifluorescence images may be acquired.

Methods of Use:

The porous mesh devices of the present disclosure may be used for creating monolayer arrays of cells or particles, which may be used for high-throughput cell sorting and analysis, or other particle sorting applications.

For example, in some instances, a monolayer of separated, single cells or particles may be transferred to another substrate, e.g., a planar glass or polymer substrate that has been configured to present a surface to which the cells or particles readily adhere. The adhered cells or particles may then be subjected to further analysis, e.g. image-based analysis for detection of specific cell surface markers.

In another example, the presently disclosed porous mesh devices may be used to pre-screen and separate cells or other particles to create a monolayer of cells or particles prior to transferring them into a microchannel plate device such as that described in U.S. Patent Application Publication No. 2018/0353960 A1. The use of the presently disclosed porous mesh devices may enable more efficient loading of the microchannel plate, and more efficient subsequent release of single cells from the microchannels by virtue of creating a monolayer of individual cells that minimizes the probability of introducing cell doublets, cell triplets, etc., into the microchannels that may cause clogging of the microchannels.

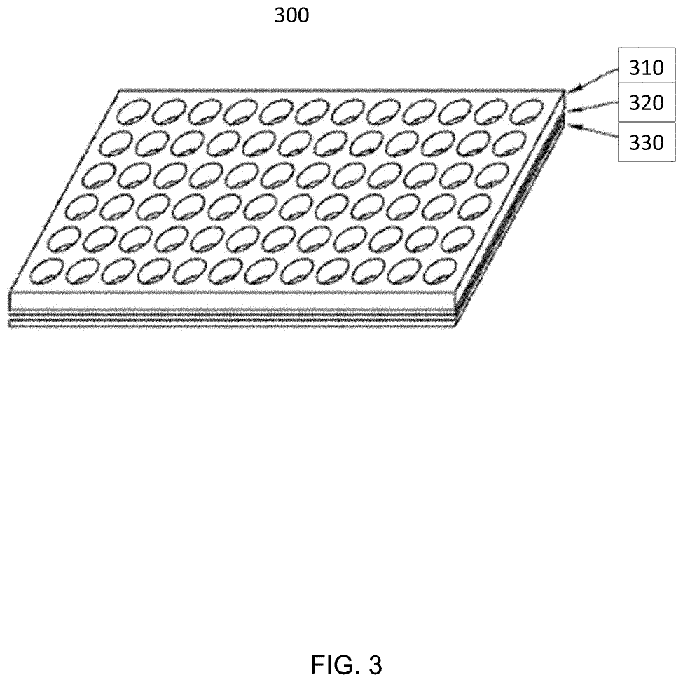

FIG. 3 illustrates components of a microchannel plate device 300 for sorting cells or particles, as described in U.S. Patent Application Publication No. 2018/0353960 A1. The portion of the device illustrated in FIG. 3 includes substrate 310, comprising a plurality of microchannels, which is adhered to a metal frame 330 by means of a seal 320.

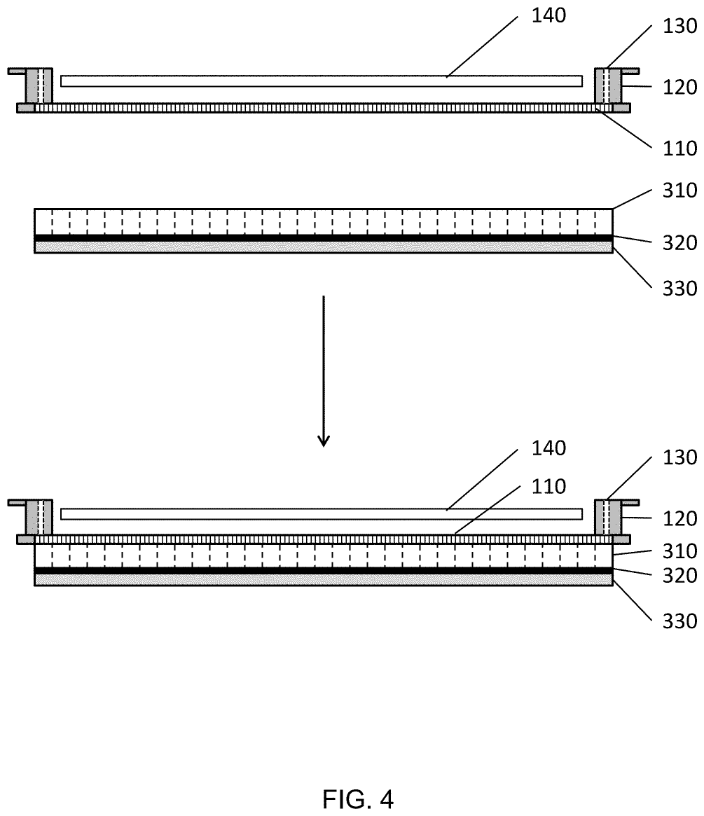

FIG. 4 illustrates the use of a porous mesh device of the present disclosure to separate cells into a monolayer which may then be transferred to a microchannel plate 310. The porous mesh is loaded by dispensing a cell suspension into fluid dispensing ports 130 integrated into support frame 120, where the fluid dispensing ports are configured such that the dispensed cell suspension flows onto the top surface of the porous mesh 110. The device may be tilted at an inclination angle of, e.g., between about 1 degree and about 10 degrees, to facilitate loading of single cells into the pores of the porous mesh by means of capillary action, as described above. The loaded mesh is then placed in contact with the top surface of the microchannel plate, as illustrated in the lower panel of FIG. 4, and suction is applied from below to draw single cells out of mesh 110 and into the microchannels of substrate 310. In some instances, the porous mesh device comprises a plate 140, as described above. In these instances, application of pressure to the top side of plate 140 results in a transfer of pressure to the cell suspension and facilitates the transport of single cells from mesh 110 into the microchannels of substrate 310.

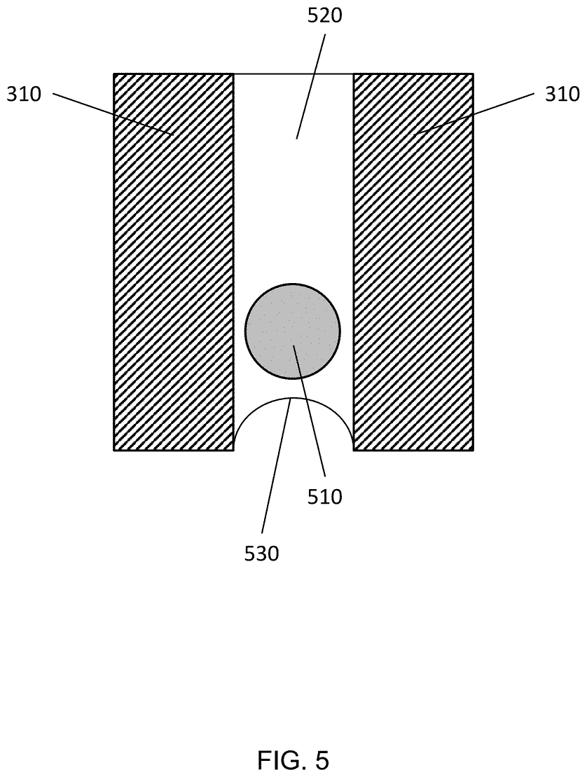

FIG. 5 illustrates a single cell 510 suspended within the fluid 520 in a microchannel of substrate 310 by means of the meniscus 530 created through surface tension and the wetting of the microchannel walls by fluid 520.

FIG. 6 illustrates the use of a porous mesh device of the present disclosure with a compressible medium 610 to separate cells. The compressible medium is applied to the mesh 110, thereby allowing fluid to transfer from the compressible medium to the mesh 110. In some instances, the compressible medium may facilitate the transfer of media and cells from the mesh 110 to the plate 140.

In some instances, the use of the disclosed porous mesh device as a pre-screening tool for loading a microchannel plate device (such as that described in U.S. Patent Application Publication No. 2018/0353960 A1) leads to more efficient loading of the microchannels with single cells or single particles. In some instances, the efficiency of loading microchannels (i.e., the percentage of microchannels comprising a single cell or particle) using the presently disclosed mesh devices may be at least 50%, at least 60%, at least 70%, at least 80%, at least 85%, at least 90%, at least 95%, at least 98%, at least 99%, or 100%.

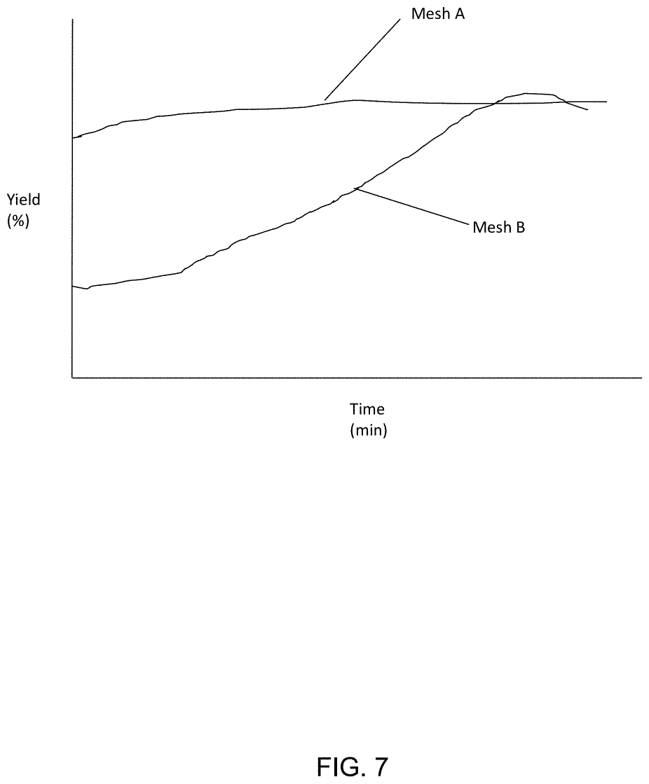

In some instances, upon loading the disclosed porous mesh device with the cell containing media, the cells or particles settle onto the plate with greater than 50%, greater than 60%, greater than 70%, greater than 80%, greater than 85%, greater than 90%, greater than 95%, greater than 98%, greater than 99% or 100% efficiency or yield (e.g., dispensed cells to visualized cells) in less than 20 minutes, less than 19 minutes, less than 18 minutes, less than 17 minutes, less than 16 minutes, less than 15 minutes, less than 14 minutes, less than 13 minutes, less than 12 minutes, less than 11 minutes, less than 10 minutes, less than 9 minutes, less than 8 minutes, less than 7 minutes, less than 6 minutes, less than 5 minutes, less than 4 minutes, less than 3 minutes, less than 2 minutes, less than 1 minutes, or less than 0 minutes. In some instances, upon loading the disclosed porous mesh device with the cell containing media, the cells or particles settle onto the plate with greater than 50%, greater than 60%, greater than 70%, greater than 80%, greater than 85%, greater than 90%, greater than 95%, greater than 98%, greater than 99% or 100% efficiency in more than 0 minutes, more than 1 minutes, more than 2 minutes, more than 3 minutes, more than 4 minutes, more than 5 minutes, more than 6 minutes, more than 7 minutes, more than 8 minutes, more than 9 minutes, more than 10 minutes, more than 11 minutes, more than 12 minutes, more than 13 minutes, more than 14 minutes, more than 15 minutes, more than 16 minutes, more than 17 minutes, more than 18 minutes, more than 19 minutes, or more than 20 minutes. Any of the lower and upper values described in this paragraph may be combined to form a range included within the present disclosure, for example, in some instances the cells or particles settle onto the plate with greater than 50%, greater than 60%, greater than 70%, greater than 80%, greater than 85%, greater than 90%, greater than 95%, greater than 98%, greater than 99% or 100% efficiency in about 0 to 3 minutes. Those of skill in the art will recognize that in some instances, the settling time may have any value within this range, e.g., about 0 minutes. FIG. 7 represents the performance of the porous mesh devices of the present disclosure. As shown in FIG. 7, both mesh A and mesh B exhibit high efficiency of total cells dispensed on the plate. Mesh A exhibits faster settling rates than mesh B.

Because of the increased single cell or single particle loading efficiency achieved through the use of the disclosed mesh devices as a pre-screen for loading microchannel plate devices (such as those described in U.S. Patent Application Publication No. 2018/0353960 A1), the use of the disclosed mesh devices may also lead to more efficient release of single cells or single particles from the microchannels using the laser-based extraction technique described in U.S. Patent Application Publication No. 2018/0353960 A1. In some instances, the efficiency of release of single cells or single particles may be at least 50%, at least 60%, at least 70%, at least 80%, at least 85%, at least 90%, at least 95%, at least 98%, at least 99%, or 100%.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

1. A particle loading system comprising:
a porous mesh having a plurality of openings arranged in a repeating pattern across a surface of said mesh, wherein said surface comprises a first surface and a second surface opposite to the first surface,
wherein the mesh is used for preparing a layer of target particles distributed and spaced apart in a two-dimensional configuration on said mesh, and
wherein each of the plurality of openings of the mesh is configured to receive and permit a target particle to pass through when a fluid containing the target particles is dispensed on the first surface of the mesh.

2. The system of claim 1, wherein the repeating pattern comprises a cross-hatch pattern.

3. The system of claim 1, wherein each of the plurality of openings is configured to receive and permit no more than one target particle to pass through from the first surface to the second surface of the mesh in a given instance.

4. The system of claim 1, wherein the layer of target particles is held together by surface tension between the fluid and the plurality of openings.

5. The system of claim 1, wherein the layer of target particles comprises one or more monolayers of cells distributed and spaced apart in the two-dimensional configuration on the mesh.

6. The system of claim 1, further comprising:
a fluid distribution apparatus configured to come in contact with the second surface of the mesh, wherein said contact distributes the fluid over distal ends of the plurality of openings to aid the preparation of the layer of target particles.

7. The system of claim 6, wherein the fluid distribution apparatus is configured to cause the layer of target particles to be held together in a layer of the fluid.

8. The system of claim 6, wherein the fluid distribution apparatus is configured to translate across the second surface of the mesh from one end of the mesh to an opposite end of the mesh.

9. The system of claim 6, wherein the second surface of the mesh is configured to translate along the fluid distribution apparatus from one end of the mesh to an opposite end of the mesh.

10. The system of claim 6, wherein a contact force between the fluid distribution apparatus and the second surface of the mesh ranges from about 0.01 N to about 0.03 N.

11. The system of claim 6, wherein the fluid distribution apparatus comprises a spreading element selected from the group consisting of a semi-cylindrical roller, a cylindrical roller, and a squeegee blade.

12. The system of claim 1, wherein the mesh is made of a flexible material.

13. The system of claim 12, wherein the mesh is held in tension by a support frame when the fluid containing the target particles is dispensed on the first surface of the mesh.

14. The system of claim 13, wherein the mesh is capable of flexing by different amounts, depending on (1) a volume of the fluid dispensed on the first surface of the mesh and (2) the tension within the mesh as provided by the support frame.

15. The system of claim 13, further comprising:
one or more dispense ports coupled to the support frame and configured to dispense the fluid containing the target particles at one or more edges of the mesh.

16. The system of claim 15, wherein the one or more dispense ports are configured to dispense the fluid containing the target particles at a selected edge of the mesh.

17. The system of claim 16, wherein the mesh is configured to be tilted at an inclination angle when the fluid containing the target particles is dispensed on the mesh.

18. The system of claim 17, wherein the mesh is tilted at the inclination angle to cause the fluid containing the target particles to move across the mesh via capillary action against gravity.

19. The system of claim 18, wherein the movement of the fluid across the mesh via the capillary action aids in reducing air bubbles within the layer of the target particles.

20.-26. (canceled)

27. The system of claim 1, further comprising a compressible medium configured to dispense fluid onto the mesh.

28.-29. (canceled)
